(19) United States
(12) Patent Application Publication
(10) Pub. No.: US 2006/0134772 A1
(43) Pub. Date: Jun. 22, 2006

(54) System for locating cells and for cellular analysis

(75) Inventors: Robin R. Miles, Danville, CA (US); Jackie Crawford, Livermore, CA (US); Allen T. Christian, Tracy, CA (US)

Correspondence Address: Eddie E. Scott;Assistant Laboratory Counsel, Lawrence Livermore National Laboratory, P.O. Box 808, L-703, Livermore, CA, 94551, US

(73) Assignee: The Regents of the University of California

(21) Appl. No.: 11/283,454

(22) Filed: Nov. 17, 2005

Related U.S. Application Data
(60) Provisional application No. 60/629,465, filed on Nov. 18, 2004.
(60) Provisional application No. 60/629,355, filed on Nov. 18, 2004.

(51) Int. Cl. C12M 1/00

(52) U.S. Cl. 435/283.1

(57) ABSTRACT

A system for locating a cell for analysis. A capture structure is provided with a flow channel in the capture structure and a capture well in the flow channel. A fluid flows along the fluid flow channel and through the capture well. The cell flows with the fluid along the flow channel into the capture well but no further. In one embodiment a capture structure is provided and a flow channel and capture well are located in the capture structure. A fluid carrying the cell flows along the fluid flow channel and into the capture well. The cell flows into the capture well but no further.

### CROSS-REFERENCE TO RELATED APPLICATIONS

This application claims the benefit of U.S. Provisional Patent Application No. 60/629,465 titled “Device for Locating Cells and Aids for Cellular Analysis” filed Nov. 18, 2004 and U.S. Provisional Patent Application No. 60/629,355 titled “Device for Locating Cells Analysis in Vertical Format” filed Nov. 18, 2004. U.S. Provisional Patent Application No. 60/629,465 titled “Device for Locating Cells and Aids for Cellular Analysis” filed Nov. 18, 2004 and U.S. Provisional Patent Application No. 60/629,355 titled “Device for Locating Cells Analysis in Vertical Format” are incorporated herein by this reference.

The United States Government has rights in this invention pursuant to Contract No. W-7405-ENG-48 between the United States Department of Energy and the University of California for the operation of Lawrence Livermore National Laboratory.

### BACKGROUND

1. Field of Endeavor

The present invention relates to cell location and more particularly to a system for locating cells for analysis and aids for cellular analysis.

2. State of Technology

A vast range of technologies has been developed to understand cell function. Technologies exist to study the damage done to a single DNA base, the levels of individual RNA transcripts in a cell and the effects of mutations in a protein on overall cellular function. Despite these advances, the properties of cells are still generally studied by examining large populations of cells. Although much of classical biochemistry has been built on this approach, there are several compelling reasons for analysis at the single cell level. Firstly, the process of lysing and pooling cellular material may destroy information about intracellular localization. There is increasing evidence that high concentration gradients are common, even in non-compartmentalized prokaryotic cells [Anderson, R. G. W.; Trends in Cell Biology 1993, 3, 69-72]. The ability to analyze intact single cells in vitro affords the ability to determine spatial inhomogeneities within a cell. Secondly, many fundamental biological processes, such as cell differentiation [Jürgens, G.; Markus Grebe, M.; Steinmann, T.; Current Opinion in Cell Biology 1997, 9(6), 849-852], carcinogenesis [Hu, K.; Ahmadzadeh, H.; Krylov, S. N.; Anal. Chem. 2004, 76(13), 3864-3866], sporulation [Pogliano, J.; Sharp, M. D.; Pogliano, K. J.; Bacteriol. 2002, 184(6), 1743-1749] or cell-cell communication [Rossant, J. Seminars in Cell & Developmental Biology 2004, 15,(5), 573-581] involve asymmetries between individual cells and aggregate measurements do not capture the basic features of such processes. The ability to perform measurements on individual cells allows precise study of such asymmetric processes.

In addition, recent studies have demonstrated high levels of both intrinsic and extrinsic variability in cloned cells under identical conditions [Elowitz, M. B.; Levine, A. J.; Siggia, E. D.; Swain, P. S.; Science 2002, 297(5584), 1183-1186]. One consequence of such intercellular variability is that many relevant cellular properties may not be reflected in average concentrations. The study of diversity at the single cell level is a large untapped field in cell biology. Finally mathematical models of metabolic and regulatory processes require experimental validation. The ability to measure biochemical responses from many individual cells provides both average responses and the variance, which is essential in determining the accuracy of the model and the fundamental limits of model precision due to intrinsic stochasticity or dependence on initial conditions.

Many of the methods being developed for analysis at the single cell level, such as in situ hybridization [Le Guellec, D.; Biol. Cell 1998, 90(4), 297-306] and fluorescence tagging of proteins [Jarvik, J. W.; Fisher, G. W.; Shi, C.; Hennen, L.; Hauser, C.; Adler, S.; Berget, P. B.; Biotechniques 2002, 33(4), 852-854, 856, 858-860 passim], provide data on spatial localization and phenotypic response in individual cells. These techniques allow observation, visualization and classification of the morphology of many types of cells at the single cell level in great detail. However, they all need to examine a sufficient number of individual cells from a given population to build statistical confidence and to analyze many parameters from each cell in order to identify and/or define variability of biological interest for this population. Therefore the prospect of maintaining single cells in an ordered array to create a sensitive, high throughput system for analyzing large numbers of single cells is of strong scientific interest.

There has been significant interest in arraying cells in recent years, as parallel single cell studies often provide a suitable model for analyzing complex interactions in basic cell biology research. A variety of methods have been proposed [Andersson, H.; van den Berg, A.; Sensors and Actuators 2003, B 92, 315-325, and Voldman; J. Nat. Mater. 2003, 2(7), 433-434]. Several microfabrication techniques exist to control single cell adhesion on the micron scale, [Thomas, C. H.; L'Hoest, J-B.; Castner, D. G.; McFarland, C. D.; Healy, K. E.; J. Biomechanical Engineering 1999, 121(1), 40-48.; Thomas, C. H.; Collier, J. H.; Sfeir, C. S.; Healy, K. E.; Proc. Nat Acad. Sci. 2002, 99(4), 1972-1977; Goessl, A.; Bowen-Pope, D. F.; Hoffman, A. S.; J. Biomedical Mater. Res. 2001, 57(1), 15-24; Michel, R.; Lussi, J. W.; Csucs, G.; Reviakine, I.; Danuser, G.; Ketterer, B.; Hubbell, J. A.; Textor, M.; Spencer, N. D.; Langmuir 2002, 18(8), 3281-3287; and Tourovskaia, A.; Barber, T.; Wickes, B. T.; Hirdes, D.; Grin, B.; Castner, D. G.; Healy, K. E.; Folch, A. F.; Langmuir 2003, 19(11), 4754-4764]. The most straightforward of these is microcontact printing, popularized by Whitesides and Ingber using alkane thiols [Singhvi, R.; Kumar, A.; Lopez, G. P.; Stephanopoulos, G. N.; Wang, D. I. C.; Whitesides, G. M.; Ingbar, D. E.; Science 1994, 264, 696-698]. Photolithographic patterning has also proven a robust patterning methodology as demonstrated by Thomas et al [Thomas, C. H.; L'Hoest, J-B.; Castner, D. G.; McFarland, C. D.; Healy, K. E.; J. Biomechanical Engineering 1999, 121(1), 40-48, and Thomas, C. H.; Collier, J. H.; Sfeir, C. S.; Healy, K. E.; Proc. Nat Acad. Sci. 2002, 99(4), 1972-1977]. Regions of adhesive islands surrounded by a durable solution polymerized, non-adhesive coating [Bearinger, J. P.; Castner, D. G.; Chen, J.; Hubchak, S.; Golledge, S. L.; and Healy, K. E.; Langmuir 1997, 13(19), 5175-5183] can maintain viable cells for up to 60 days. Photolithography combined with plasma polymerization of tetraglyme can also control shape and size of cells. More recently, Michel et al. [Michel, R.; Lussi, J. W.; Csucs, G.; Reviakine, I.; Danuser, G.; Ketterer, B.; Hubbell, J. A.; Textor, M.; Spencer, N. D.; Langmuir 2002, 18(8), 3281-3287] introduced substrate dependent directed self assembly (SMAP) for fabrication of biologically relevant chemical patterns for single cells and Tourovskaia et al. introduced stencil directed oxygen plasma to selectively remove non-adhesive chemistry and therefore allow micropatterning of cells [Tourovskaia, A.; Barber, T.; Wickes, B. T.; Hirdes, D.; Grin, B.; Castner, D. G.; Healy, K. E.; Folch, A. F.; Langmuir 2003, 19(11), 4754-4764]. While these techniques are all valuable for isolation of adhesive cells, they cannot be used to study cells that natively culture in suspension, such as chondrocytes or non-adhesive cells, such as leucocytes.

Microfluidic cell trapping that does not rely on surface chemistry, such as optical tweezers, magnetic activated cell sorting, filtration, and electric field-based manipulation platforms, have also been developed [Wheeler, A.; Throndset, W.; Whelan, R.; Leach, A.; Zare, R.; Liao, Y.; Farrell, K.; Manger, I.; Daridon, A.; Anal. Chem. 2003, 75 (14), 3581-3586; Pedersen, S.; Kutchinsky, S.; Friis, S.; Krzywkkowski, K.; Tracy, C.; Vestergaard, R.; Sorensen, C.; Vennerberg, H.; Taboryski, R.; Digest of Technical Papers, Transducers '03, Boston MA, June 2003; pp 1059-1062; Schnelle, T.; Muller, T.; Reiochle, C.; Fuhr, G.; App. Physics B. 2000, 70, 267-274; Voldman, J.; Gray, M.; Toner, M.; Schmidt, M.; Anal. Chem. 2002, 74, 3984-3990; and Pethig, R.; Huang, Y.; Wang, X-B.; Burt, J.; J. Phys. D; Applied Physics 1992, 24, 681-688]. Dielectrophoretic trapping is attractive at the microscale (<1 mm) because the forces achieved through patterning of electrodes and the application of an electric field in microfluidic devices are sufficient to manipulate cells at comparable distances [Pohl, H. A.; Dielectrophoresis, Cambridge University Press: New York, N.Y., 1978; Jones, T.; Electromechanics of Particles, Cambridge University Press: New York, N.Y., 1995; Schnelle, T.; Hagedorn, R.; Fuhr, G.; Fiedler, S.; Muller, T.; Biochim. Biophys. Acta 1993, 1157(2), 127-140; Muller, T.; Pfenning, A.; Klein, P.; Gradl, G.; Jager, M.; Schnelle, T.; IEEE Engineering in Medicine and Biology Magazine, November/December 2003, 51-61; Gascoyne, P. R. C.; Vykoukal, J.; Electrophoresis, 2002, 23, 1973-1983; and Cummings, E.; Singh, A.; Anal. Chem. 2003, 75, 4724-4731]. While quadrupole electrode configurations have been used to trap single cells, the majority of devices trap multiple cells either along periphery of the electrodes or between them depending on the electrical properties of the particle and the media and the applied excitation frequency. Use of dielectrophoretic techniques require that the system design take into account the relative complex permitivities of the media and the cells to ensure that sufficient force is generated.

Trapping cells mechanically on a microchip poses challenges primarily due to fluidic stresses and cell fragility. Although not optimized for single cells, Chin et al [Chin, V. I.; Taupin, P.; Sanga, S.; Scheel, J.; Gage, F. H.; Bhatia, S. N.; Biotech. Bioeng. 2004, 88 (3), 399-415] microfabricated an array of wells of various heights on cover slips to study stem cell proliferation. A multiple patch clamp array chip was described by Seo et al [Seo J.; Ionescu-Zanetti C.; Diamond J.; Lal R.; Lee L. P.; App. Phys. Lett. 2004, 84 (11): 1973-1975] and parallel gene transfection into arrayed cells has been reported by Tixier-Mita et al [Tixier-Mita, A.; Jun, J.; Ostrovidov, S.; Chiral, M.; Frenea, M.; Le Pioufle B.; Fujita, H.; Proceedings of MicroTAS, Malmö, Sweden September 2004 (in press)]. Yang et al. configured a cell docking system that accommodated single cells lining a wall along a dam [Yang, M.; Li, C.; Yang, J.; Anal. Chem. 2002, 74, 3991-4001]. Embryo transportation and retention in microfluidic wells has been demonstrated by Glasgow et al. [Glasgow I. K; Zeringue H. C; Beebe D. J; Choi S. J; Lyman J. T; Chan N. G; Wheeler M. B.; IEEE Trans. Biomed. Eng. 2001, 48 (5), 570-578].

### SUMMARY

It is advantageous to position and hold cells in an array format in order to perform cellular quantitative analyses. In this way, the effects of certain tests can be attributed to specific cells rather in random batch mode as is typical today. The present invention provides a system for capturing and holding cells. The present invention provides a system for probing cells to perform certain types of analyses.

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

The present invention comprises a system for locating a cell for analysis. A capture structure is provided with a flow channel in the capture structure and a capture well in the flow channel. A fluid flows along the fluid flow channel and through the capture well. The cell flows with the fluid along the flow channel into the captures well but no further. One embodiment of the present invention provides an apparatus for capturing a cell for analysis comprising a capture structure, a flow channel in the capture structure, a capture well in the flow channel, and a fluid, wherein the fluid carries the cell so that the cell is captured in the capture well for analysis.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1 illustrates a capture well device constructed in accordance with one embodiment of the present invention.

FIG. 2 is a top view of the capture well device shown in FIG. 1.

FIG. 3 illustrates another embodiment of a capture well device of the present invention.

FIG. 4 illustrates yet another embodiment of a capture well device of the present invention.

FIG. 5 is another embodiment of a capture well device of the present invention.

FIG. 6 is another embodiment of a capture well device of the present invention.

FIG. 7 is a top view of the capture well device.

FIG. 8 illustrates another embodiment of a capture well device of the present invention.

FIG. 9 illustrates another embodiment of a capture well device constructed in accordance with the present invention.

FIG. 10 is a top view of the capture well device is shown with the cover removed.

FIG. 11 illustrates another embodiment of a capture well device of the present invention.

FIG. 12 illustrates another embodiment of a capture well device of the present invention.

FIG. 13 illustrates another embodiment of a capture well device of the present invention.

FIG. 14 illustrates another embodiment of a capture well device of the present invention.

FIG. 15 illustrates another embodiment of a capture well device of the present invention.

### DETAILED DESCRIPTION OF THE INVENTION

Referring to the drawings, to the following detailed description, and to incorporated materials, detailed information about the invention is provided including the description of specific embodiments. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

Certain biological questions cannot be answered using current techniques of studying cells in random batch mode. Currently, a population of cells will be subjected to a certain environmental stimulus and statistics will be gathered about how many reacted in what way to the change. Minor differences between the cells cannot be accounted for using the current techniques nor can specific individual reactions be studied to find the true cause of variations in reactions between individuals within the population. To gain this knowledge, cells must be studied on an individual basis but in populations large enough for statistical relevance.

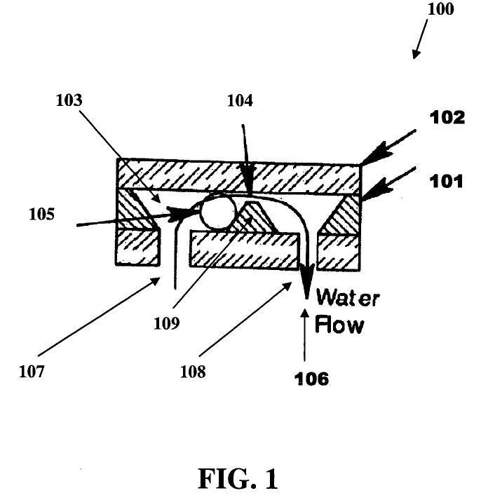

Referring now to FIG. 1, a horizontal capture well device constructed in accordance with one embodiment of the present invention is shown. This embodiment of a horizontal capture well device is designated generally by the reference numeral 100. FIG. 1 is a top view of the capture well device 100 showing a horizontal slit 104. The horizontal capture well device 100 includes a capture structure 101, a cover 102, and a flow channel 103. A horizontal slit or gap 104 is provided in the flow channel 103. As illustrated by FIG. 1, a cell 105 is introduced into the channel 103 and is swept along with the fluid 106 from a fluid inlet 107 towards a fluid outlet 108. The small gap 104 is located along the flow channel 103 such that the fluid 106 but not the cell 105 is able to traverse the gap 104. The cell 105 is captured within the structure 109 of the flow channel 103 that forms the gap 104.

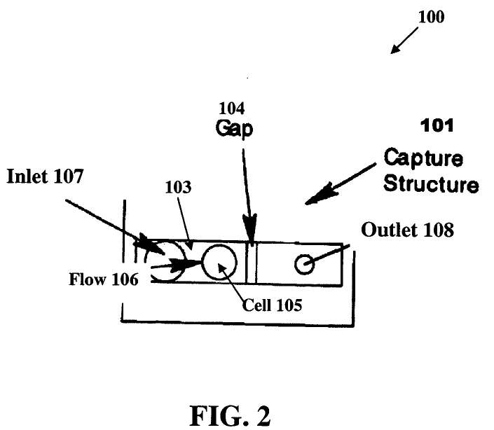

Referring now to FIG. 2, a view of the horizontal capture well device 100 is shown with the cover 102 removed. The flow channel 103 is contained in capture structure 101 and the slit or gap 104 is located in the flow channel 103. As illustrated by FIG. 2, a cell 105 is introduced into the flow channel 103 and is swept along with the fluid from a fluid inlet 107 towards a fluid outlet 108. The small gap 104 is located along the flow channel 103 such that the fluid 106 but not the cell 105 is able to traverse the gap 104. The cell 105 is captured in the gap 104.

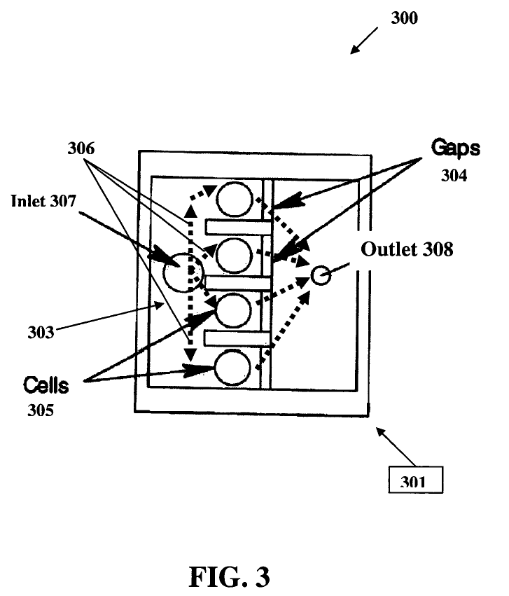

Referring now to FIG. 3, another embodiment of a horizontal capture well device of the present invention is shown. This embodiment of a horizontal capture well device is designated generally by the reference numeral 300. FIG. 3 is a top view that shows a capture device that captures an array of cells. The horizontal capture well device 300 includes a capture structure 301, a cover 302 (not shown), and a flow channel 303. FIG. 3 is a top view of the horizontal capture well device 300 with the cover 302 removed. A multiplicity of horizontal slits or gaps 304 are provided in the flow channel 303. As illustrated by FIG. 3, cells 305 are introduced into the flow channel 303 and are swept along with the fluid 306 from a fluid inlet 307 toward a fluid outlet 308. The small gaps 304 are located along the flow channel 303 such that the fluid 306 but not the cells 305 are able to traverse the gaps 304. The cells 305 are captured in the gaps 304 within the flow channel 303.

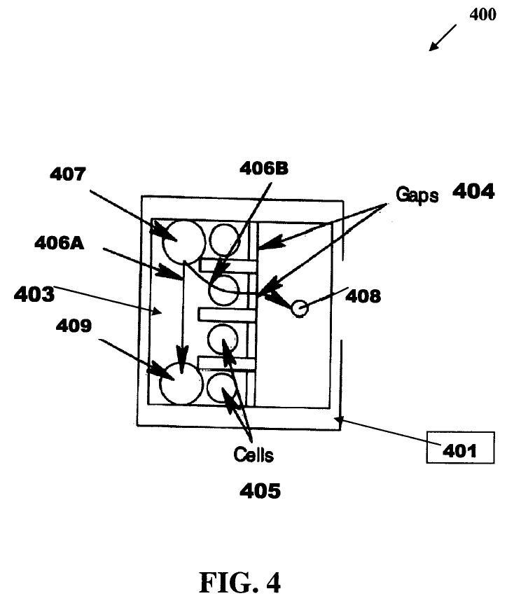

Referring now to FIG. 4, another embodiment of a horizontal capture well device of the present invention is shown. This embodiment of a horizontal capture well device is designated generally by the reference numeral 400. FIG. 4 shows a horizontal capture device that captures an array of cells 405 from a media flow 406A. The horizontal capture well device 400 includes a capture structure 401, a cover 402 (not shown), and a flow channel 403. FIG. 4 is a top view of the horizontal capture well device 400 with the cover 402 removed. A multiplicity of horizontal slits or gaps 404 are provided in the flow channel 403. As illustrated by FIG. 4, cells 405 are introduced into the flow channel 403 and are swept along with the media flow 406A from an inlet 407 toward media flow outlet 409. A portion of the flow 406B is drawn to a flow outlet 408 that provides aspiration of the cells 405 to the gaps 404. The small gaps 404 are located along the flow channel 403 such that the fluid 406B but not the cells 405 are able to traverse the gaps 404. The cells 405 are captured in the gaps 404 within the flow channel 403. For long-term studies, the additional outlet 409 allows nutrients to flow past the cells 405 while maintaining a slight amount of flow 406B through the gaps 404 to hold the cells 405.

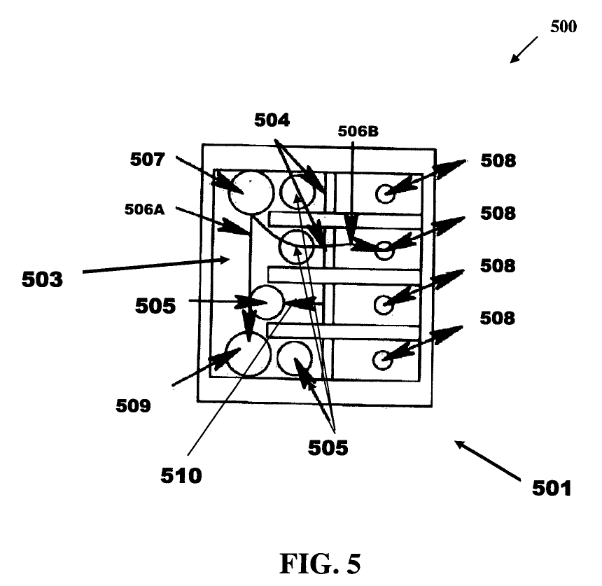

Referring now to FIG. 5, another embodiment of a horizontal capture well device of the present invention is shown. This embodiment of a horizontal capture well device is designated generally by the reference numeral 500. FIG. 5 shows a horizontal capture device that captures an array of cells 505 from a media flow 506A. The horizontal capture well device 500 includes a capture structure 501, a cover 502 (not shown), and a flow channel 503. FIG. 5 is a top view of the horizontal capture well device 500 with the cover 502 removed. A multiplicity of horizontal slits or gaps 504 are provided in the flow channel 503. As illustrated by FIG. 5, cells 505 are introduced into the flow channel 503 and are swept along with the media flow 506A from an inlet 507 toward media flow outlet 509. A portion of the flow 506B is drawn to a flow outlet 508 that provides aspiration of the cells 505 to the gaps 504. The small gaps 504 are located along the flow channel 503 such that the fluid 506B but not the cells 505 are able to traverse the gaps 504. The cells 505 are captured in the gaps 504 within the flow channel 503. For long-term studies, the additional outlet 509 allows nutrients to flow past the cells 505 while maintaining a slight amount of flow 506B through the gaps 504 to hold the cells 505. The individual aspiration outputs 508 are provided for each cell unit in the array to permit backflow (illustrated by the arrow 510) over the gap 504 to force the cell 505 out of the gap 504 if it is desired to study a particular cell in another apparatus.

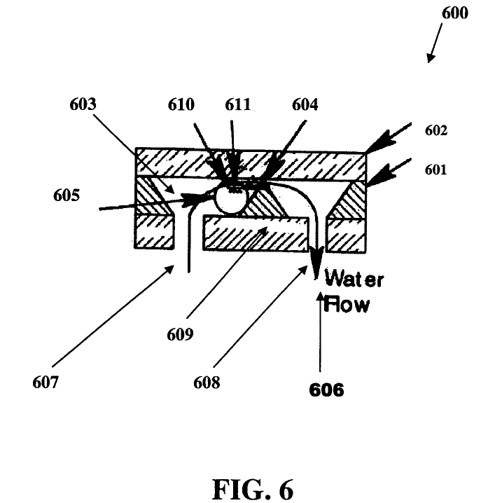

Referring now to FIG. 6, another embodiment of a horizontal capture well device constructed in accordance with the present invention is shown. This embodiment of a horizontal capture well device is designated generally by the reference numeral 600. FIG. 6 shows a horizontal capture device wherein a horizontal slit is used. The horizontal capture well device 600 includes a capture structure 601, a cover 602, and a flow channel 603. A horizontal slit or gap 604 is provided in the flow channel 603. As illustrated by FIG. 6, a cell 605 is introduced into the channel 603 and is swept along with the fluid 606 from a fluid inlet 607 towards a fluid outlet 608. The small gap 604 is located along the flow channel 603 such that the fluid 606 but not the cell 605 is able to traverse the gap 604. The cell 605 is captured within the structure 609 of the flow channel 603 that forms the gap 604. A solid probe 610 is positioned in the flow channel 603 proximate the gap 604 such that the cell 605 impales itself on the probe 610 as the cell 605 is being sucked towards the gap 604. The probe 610 is coated with a metal 611 such that surface enhanced spectroscopy can be achieved. The probe 610 is functionalized such that certain molecular interactions of interest can be studied after the probe 610 is inside the cell 605.

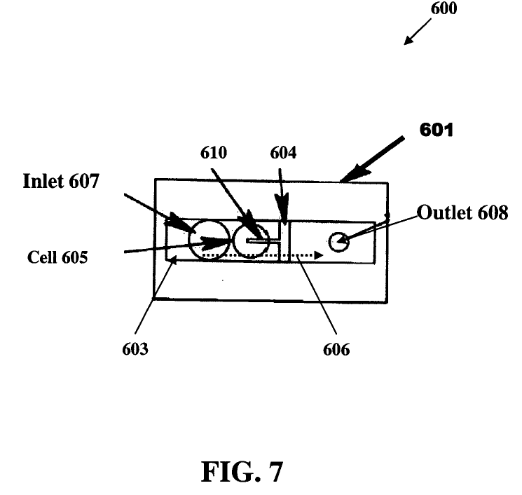

Referring now to FIG. 7, a top view of the horizontal capture well device 600 is shown with the cover 602 removed. The flow channel 603 is contained in capture structure 601 and the slit or gap 604 is located in the flow channel 603. As illustrated by FIG. 7, a cell 605 is introduced into the flow channel 603 and is swept along with the fluid from a fluid inlet 607 towards a fluid outlet 608. The small gap 604 is located along the flow channel 603 such that the fluid 606 but not the cell 605 is able to traverse the gap 604. The cell 605 is captured in the gap 604. The probe 610 is positioned in the flow channel 603 proximate the gap 604 such that the cell 605 impales itself on the probe 610 as the cell 605 is being sucked towards the gap 604. The probe 610 is functionalized such that certain molecular interactions of interest can be studied after the probe 610 is inside the cell 605.

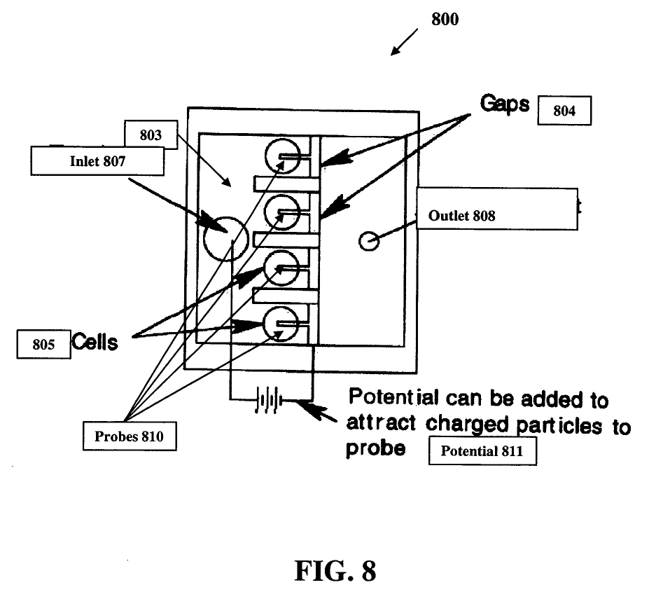

Referring now to FIG. 8, another embodiment of a horizontal capture well device of the present invention is shown. This embodiment of a horizontal capture well device is designated generally by the reference numeral 800. FIG. 8 shows a horizontal capture device that captures an array of cells and utilizes a potential 811 to attract charged particles to a probe 810. The horizontal capture well device 800 includes a capture structure 801, a cover 802 (not shown), and a flow channel 803. FIG. 8 is a top view of the horizontal capture well device 800 with the cover 802 removed. A multiplicity of horizontal slits or gaps 804 are provided in the flow channel 803. As illustrated by FIG. 8, cells 805 are introduced into the flow channel 803 and are swept along with the fluid 806 from a fluid inlet 807 toward a fluid outlet 808. The small gaps 804 are located along the flow channel 803 such that the fluid 806 but not the cells 805 are able to traverse the gaps 804. The cells 805 are captured in the gaps 804 within the flow channel 803. Probes 810 are positioned in the flow channel 803 proximate the gaps 804 such that the cells 805 impale themselves on the probes 810 as the cells 805 are being sucked towards the gaps 804. The probes 810 are functionalized such that certain molecular interactions of interest can be studied after the probes 810 are inside the cells 805. The probes 810 or its coating can be electrically conductive such that charged particles such as DNA will be attracted toward it. A potential can be placed on the probes relative to upstream. The charged particles can collect on the surface of the probes 810. The cells would then be impaled on the probes 610 and the field released, resulting in an injection of the charged molecules into the device 800.

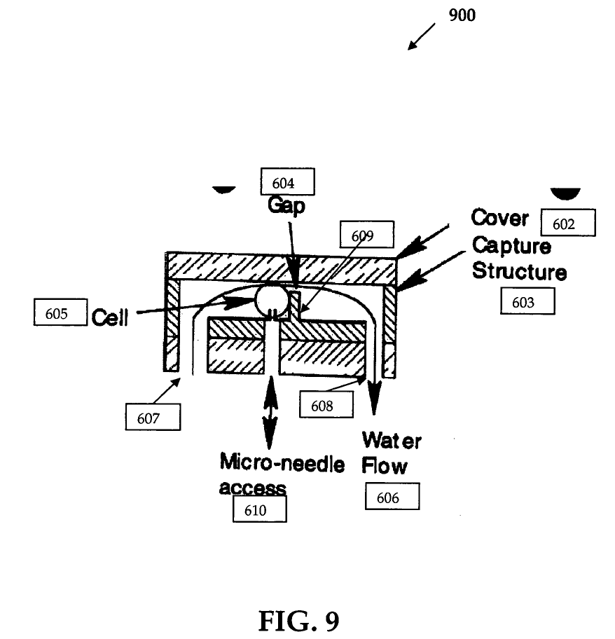

Referring now to FIG. 9, another embodiment of a horizontal capture well device constructed in accordance with the present invention is shown. This embodiment of a horizontal capture well device is designated generally by the reference numeral 900. A needle 910 is added to the structure such that injection and extraction of particles can be made to the cell 905. The cell 905 will slide into the needle 910 in a transverse method. A pressure drop across the gap 904 is optimized by adjusting the flow length of the gap 904. FIG. 9 shows a horizontal capture device wherein a horizontal slit is used. The horizontal capture well device 900 includes a capture structure 901, a cover 902, and a flow channel 903. A horizontal slit or gap 904 is provided in the flow channel 903. As illustrated by FIG. 9, a cell 905 is introduced into the channel 903 and is swept along with the fluid 906 from a fluid inlet 907 towards a fluid outlet 908. The small gap 904 is located along the flow channel 903 such that the fluid 906 but not the cell 905 is able to traverse the gap 904. The cell 905 is captured within the structure 909 of the flow channel 903 that forms the gap 904. A micro-needle 910 is positioned in the flow channel 903 proximate the gap 904 such that the cell 905 impales itself on the micro-needle 910 as the cell 905 is being sucked towards the gap 904. The micro-needle 910 is coated with a metal 911 such that surface enhanced spectroscopy can be achieved. The micro-needle 910 is functionalized such that certain molecular interactions of interest can be studied after the micro-needle 910 is inside the cell 905.

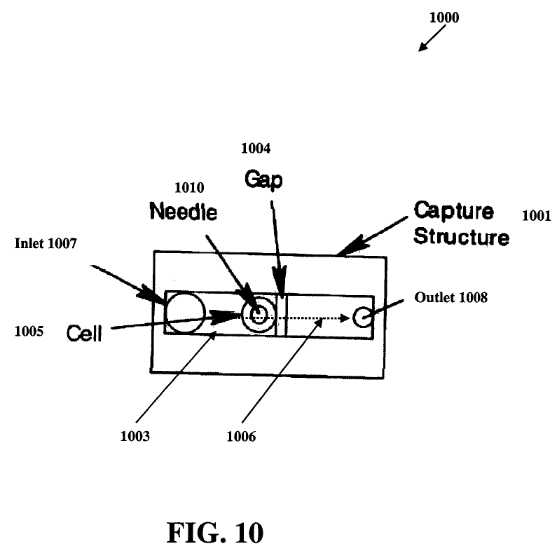

Referring now to FIG. 10, a top view of the horizontal capture well device 1000 is shown with the cover 1002 removed. The flow channel 1003 is contained in capture structure 1001 and the slit or gap 1004 is located in the flow channel 1003. As illustrated by FIG. 10, a cell 1005 is introduced into the flow channel 1003 and is swept along with the fluid from a fluid inlet 1007 towards a fluid outlet 1008. The small gap 1004 is located along the flow channel 1003 such that the fluid 1006 but not the cell 1005 is able to traverse the gap 1004. The cell 1005 is captured in the gap 1004. The needle 1010 is positioned in the flow channel 1003 proximate the gap 1004 such that the cell 1005 impales itself on the needle 1010 as the cell 1005 is being sucked towards the gap 1004. The needle 1010 is functionalized such that certain molecular interactions of interest can be studied after the needle 1010 is inside the cell 1005.

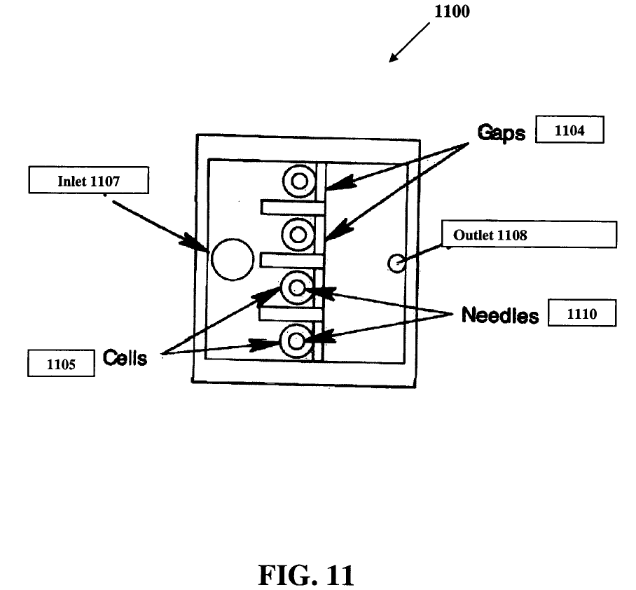

Referring now to FIG. 11, another embodiment of a horizontal capture well device of the present invention is shown. This embodiment of a horizontal capture well device is designated generally by the reference numeral 1100. FIG. 11 shows a horizontal capture device that captures an array of cells and utilizes a potential 1111 to attract charged particles to a needle 1110. The horizontal capture well device 1100 includes a capture structure 1101, a cover 1102 (not shown), and a flow channel 1103. FIG. 11 is a top view of the horizontal capture well device 1100 with the cover 1102 removed. A multiplicity of horizontal slits or gaps 1104 are provided in the flow channel 1103. As illustrated by FIG. 11, cells 1105 are introduced into the flow channel 1103 and are swept along with the fluid 1106 from a fluid inlet 1107 toward a fluid outlet 1108. The small gaps 1104 are located along the flow channel 1103 such that the fluid 1106 but not the cells 1105 are able to traverse the gaps 1104. The cells 1105 are captured in the gaps 1104 within the flow channel 1103. Needles 1110 are positioned in the flow channel 1103 proximate the gaps 1104 such that the cells 1105 impale themselves on the needles 1110 as the cells 1105 are being sucked towards the gaps 1104. The needles 1110 are functionalized such that certain molecular interactions of interest can be studied after the needles 1110 are inside the cells 1105. The needles 1110 or its coating can be electrically conductive such that charged particles such as DNA will be attracted toward it. A potential can be placed on the needles relative to upstream. The charged particles can collect on the surface of the needles 1110. The cells would then be impaled on the needles 610 and the field released, resulting in an injection of the charged molecules into the device 1100.

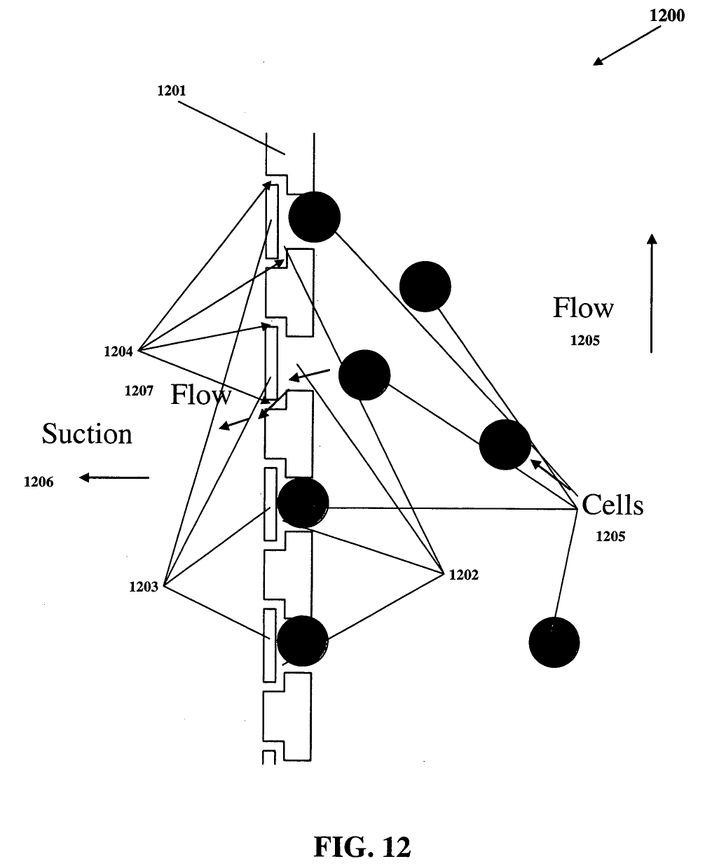

Referring now to FIG. 12, a cell-array device constructed in accordance with another embodiment of the present invention is shown. This embodiment of a cell-array device is designated generally by the reference numeral 1200. The cell-array device 1200 includes a chip 1201. A multiplicity of capture wells 1202 is located in the chip 1201. The capture wells 1202 extend into the chip 1201. The capture wells 1202 have a bottom portion 1203 and side flow passages 1204. Fluid flow channels lead to the capture wells 1202. Fluid flow 1205 is provided along the main channel and fluid flow 1207 is directed into fluid flow channels through the capture wells 1202 in the chip 1201. A source of suction 1206 brings the cells 1205 into the capture wells 1202. The cells 1206 flow with the fluid along the flow channel into the capture wells 1202 but no further. The cells are retained in the capture wells 1202 by the bottoms 1203 of the capture wells 1202. The fluid flow 1207 along the fluid flow channel and through the capture wells 1202 in the chip 1201 results in the cells 1205 being directed onto the capture wells 1202 to locate the cells for analysis.

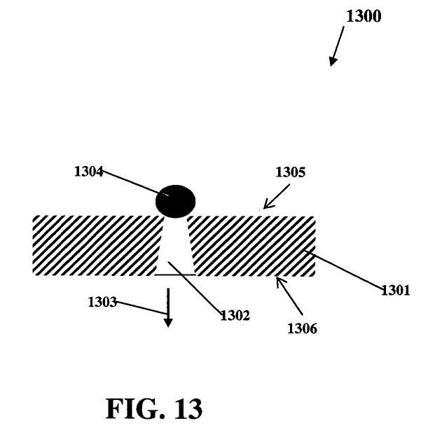

Referring now to FIG. 13, a cell-array device constructed in accordance with another embodiment of the present invention is shown. This embodiment of a cell-array device is designated generally by the reference numeral 1300. The cell-array device 1300 includes a chip 1301 with a capture well 1302 extending through the chip 1301. The font-side 1305 and back-side 1306 of the chip are identified. A suction 1303 is applied to the capture well 1302. A cell 1304 is shown in the capture well 1302.

In general, the cell 1304 is introduced into the chip 1301 through fluidic flow created by the suction 1303. The cell 1304 flows along a flow channel until it is captured in the cell capture well 1302. The mechanism for capturing the cell is though a small amount of suction flow through a small hole 1302 large enough for fluid flow but smaller than the cell 1304. The cell 1304 follows streamlines into the capture well 1302 but no further.

FIG. 13 shows a case of vertical flow wherein a small hole 1302 is used. The cell 1304 is aspirated onto the small hole 1302 which is used to locate it. The pressure drop across the gap is reduced by minimizing the flow length of the gap.

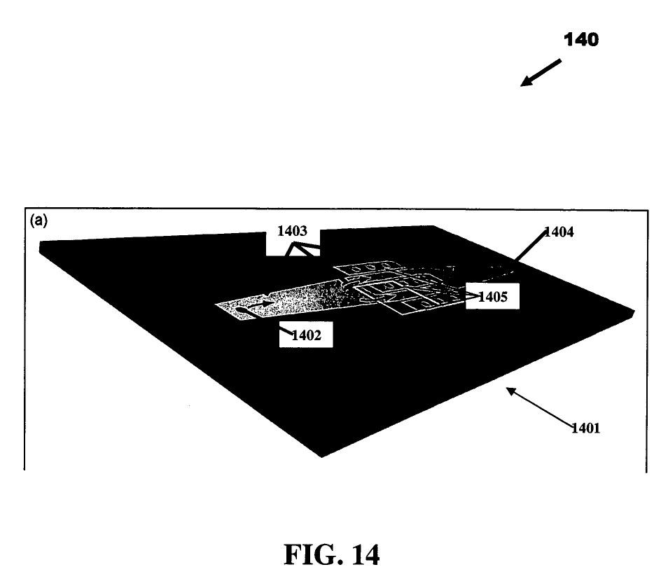

Referring now to FIG. 14, a schematic of a cell-array device constructed in accordance with another embodiment of the present invention is shown. This embodiment of a cell-array device is designated generally by the reference numeral 1400. FIG. 14 is an illustration shows vertical suction holes 1405 arranged in an array 1401 within a flow device 1400. In general, cells are introduced into the chip 1401 through fluidic flow channels. The cells flow along this channel until they are captured in one of the cell capture areas. The mechanism for capturing the cell is though a small amount of suction flow through a small hole large enough for fluid flow but smaller than the cell. The cells follow the streamlines into the capture well but no further.

The cell-array device 1400 is formed from a wafer 1401. The wafer 1401 has dimensions of 10×20 mm. Cells are carried by media flow from an inlet 1402 to an outlet 1404 as illustrated by the arrows 1403. The cells are directed into specific cell capture wells 1405.

An individual cell flows along a flow channel until it is captured in an individual cell capture well 1405. The mechanism for capturing the cell is though a small amount of suction flow through a small hole 1405 large enough for fluid flow but smaller than the cell. The cell follows streamlines into the capture well 1405 but no further. As illustrated in FIG. 14, the capture wells 1405 provide vertical flow. The cell is aspirated onto the small hole 1405 which is used to locate it. The pressure drop across the gap is reduced by minimizing the flow length of the gap. The small hole 1405 is large enough for fluid flow but smaller than the cell.

The cell-array device 1400 is comprised of a silicon wafer 1401 sandwiched between two glass plates. The silicon wafer 1401 is etched to produce several three-dimensional features that serve as media flow channels. Connections to the platform are made through the bulk of the silicon and through ports in the bottom glass plate. In this configuration the cell media sample flows through an entrance port 1402 into a main channel which branches into two identical flow channels that subsequently merge before exiting the platform through an exit port 1404. Each of the two flow channels is 4.5 mm long, 0.5 mm wide and 15 μm deep. Thirty-nine individual cell capture wells 1405, spaced at increments of 0.1 mm, line each side of the two flow channels yielding a total of 156 wells. Each well has length, width and depth dimensions of 10-20 μm and is sized to contain one cell. At the back of each individual well is a small microchannel of dimensions 4 μm wide, 15 μm long and 1.5-3.5 μm deep. These microchannels lead to a reservoir that contains a port to which suction can be applied.

Cells are arrayed in the array 1400 via a combination of capillary and pressure driven flow. Cells suspended in media are introduced to the array 1400 via the use of a pressure driven flow that results in cells suspended in the media to flow primarily through the two branches of the main channel. However, media can also flow through the microchannels at the back of the cell capture wells using capillary forces and a pressure differential when suction is applied to the ports in the reservoirs. Cells can be carried by the media flow into the wells but cannot proceed into the microchannels if the cells have larger spatial dimensions than the microchannels.

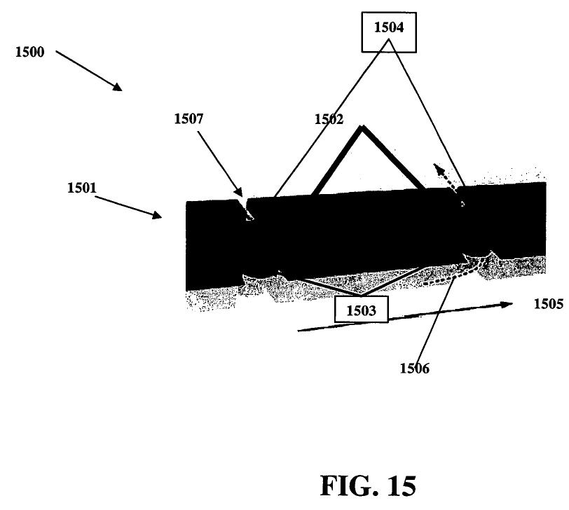

Referring now to FIG. 15, another embodiment of a cell-array device constructed in accordance with one embodiment of the present invention is shown. This embodiment of a cell-array device is designated generally by the reference numeral 1500. The cell-array device 1500 includes a chip 1501 with capture wells 1502 extending through the chip 1501. The capture wells include a recessed section 1503 and a small hole 1507. A suction is applied to fluid causing the fluid to flow through the capture wells 1502. Cells 1504 are shown in the capture wells 1502.

In general, the cells 1504 are introduced into the chip 1501 through fluidic flow created by suction. The flow of fluid is illustrated by the arrow 1505. The cell 1504 flows along a flow channel as illustrated by the second arrow 1506 until the cell 1504 is drawn into and is captured in the cell capture well 1502. The mechanism for capturing the cell is though a small amount of suction flow through the small hole 1507 large enough for fluid flow but smaller than the cell 1504. The cell 1504 follows streamlines into the capture well 1502 but no further.

Schematics of embodiments of cell-array devices constructed in accordance with the present invention have been shown in FIGS. 1 through 15. In general, cells are introduced into the device through fluidic flow channels. The cells flow along this channel until they are captured in one of the cell capture pits. The mechanism for capturing the cell is through a small amount of suction flow through a small hole or slit large enough for fluid flow but smaller than the cell. The cells follow the streamlines into the capture well but no further.

The cell-array devices are comprised of a silicon wafer sandwiched between two glass plates. The silicon wafer is etched to produce several three-dimensional features that serve as media flow channels. Connections to the platform are made through the bulk of the silicon and through ports in the bottom glass plate. In this configuration the cell media sample flows through an entrance port into a main channel which branches into two identical flow channels that subsequently merge before exiting the platform through an exit port. Each of the two flow channels is 4.5 mm long, 0.5 mm wide and 15 μm deep. Thirty-nine individual cell capture wells 405, spaced at increments of 0.1 mm, line each side of the two flow channels yielding a total of 156 wells. Each well has length, width and depth dimensions of 10-20 μm and is sized to contain one cell. Other embodiments of the present invention use smaller sized wells. In the other embodiments, the smaller sized wells are 1-4 μn that can be used to array bacteria. At the back of each individual well is a small microchannel of dimensions 4 μm wide, 15 μm long and 1.5-3.5 μm deep. These microchannels lead to a reservoir that contains a port to which suction can be applied.

Cells are arrayed in the array via a combination of capillary and pressure driven flow. Cells suspended in media are introduced to the array via the use of a pressure driven flow that results in cells suspended in the media to flow primarily through the two branches of the main channel. However, media can also flow through the microchannels at the back of the cell capture wells using capillary forces and a pressure differential when suction is applied to the ports in the reservoirs. Cells can be carried by the media flow into the wells but cannot proceed into the microchannels if the cells have larger spatial dimensions than the microchannels.

Cell Capture Platform Construction—The etched silicon wafer containing the flow channels is designed and fabricated using standard microfabrication techniques. Flow access ports are etched in one side of the wafer using KOH wet etches and the flow channels for the cell array are etched in the opposing side using reactive ion etching (RIE). Initially, 300 μm thick silicon wafers are coated with silicon nitride using a low-pressure chemical vapor deposition process. The bottom side of the wafer is photolithographically patterned for the first KOH etch and the silicon nitride etched to approximately half the original thickness using RIE. The bottom side of the wafer is photolithographically patterned for the second KOH etch and the silicon nitride etched using RIE until the silicon nitride is completely removed from the areas delineated for the first KOH etch. The silicon wafer is etched using a 40% KOH solution at 85° C. to a depth of about 200 μm. The silicon nitride is then etched so that the areas defining the second KOH etch are removed. The silicon is then etched about 90 μm. The second etch establishes the manifold area for the reservoirs and suction ports. The silicon nitride is removed from the wafer and the top of the wafer photolithographically patterned for the microchannels. The channels are etched using deep RIE (Surface Technology Systems, Imperial Park, Newport, United Kingdom) to a depth ranging from 1 to 4 μm. The resist (Shipley Phoenix, Ariz.) was used as the etch mask. Following this etch, resist was removed and new resist spun over the wafer covering the shallow etch features. The larger flow channels were photolithographically patterned and etched using the deep RIE etch to a depth of about 15 μm. During this etch the flow paths defined by the KOH and the RIE etches co-join. Glass plates are then anodically bonded to the silicon wafer and the composite diced into individual cell capture arrays. Prior to bonding flow holes are drilled through the glass plates to access the silicon flow channels.

The cell capture array is placed over a copper ring. The copper ring acts as a thermal reservoir that maintains a constant temperature of 37° C. within the flow channels of the array. The ring is heated resistively using three 100-ohm resistors powered through a temperature controller (Omega Instruments model CNi 1633, Stamford, Conn.) with temperature sensed using a resistance temperature detector (RTD) in contact with the ring. Flow input and output lines to the array consist of polyetheretherketone (PEEK) flow tubes press-sealed around the flow ports using specialized elastomeric o-rings. To provide structural integrity the assembly of the array, copper ring and PEEK tubing are placed within a 13 cm diameter circular delrin (polyoxymethylene) platform that sits in an acrylic base that is designed for easy mounting to an x-y table of a microscope. The assembly is locked in place within the platform using a metal ring while the PEEK tubing exits from the backside of the platform. The platform is readily placed under most optical microscopes enabling detailed imaging of the arrayed cells via reflected light.

Cells and media are introduced and suction applied to the platform via the use of syringes coupled to polypropylene tubing. This tubing is coupled to the platform's PEEK tubing using elastomeric seal rings. Media is perfused through the system using a syringe pump (Cole-Palmer model 74900, Vernon Hills, Ill.) controlling a 3 ml syringe. A syringe injection port (Upchurch Scientific V322, Oak Harbor, Wash.) is used to introduce cells, dyes, stains, nanoparticles and biomolecular constructs to the array. A reservoir is used to collect waste downstream of the platform. A shut off valve (Upchurch, P782, Oak Harbor, Wash.) on the suction port outlet, and a syringe downstream of this port are used to apply suction to array cells.

Cell Loading Protocol—Prior to use, all tubing and the platform is rinsed using a 70% ethanol solution. The tubing is then rinsed with approximately 3 ml of sterile water. Approximately 2.5 ml of media (0.1% serum) pre-saturated in an incubator to 5% CO₂ at 37° C. is aspirated into a 3 ml volume media syringe. Small bubbles are often observed along the syringe walls presumably due to nucleation of dissolved gasses in the media. These bubbles prove difficult to remove and are often not removed. The media syringe is placed in the media syringe pump and the inlet and suction valves to the platform are opened allowing flow from the syringe into the platform. The media syringe pump is elevated above the platform to discourage bubbles nucleated within the syringe from entering the tubing. Prior to cell loading the inlet valve immediately down stream of the media syringe is closed and cells suspended in media are injected into the platform using the syringe injection port. When cells are seen to be flowing through the array, the cell injection is halted and the flow allowed to slow. The suction syringe is aspirated 2-3 times to capture cells in the wells and the suction syringe valve is then closed. Following cell capture, the inlet valve immediately downstream of the media syringe pump is reopened and the media syringe pump is set to dispense media at the rate of 0.5 μl/min through the platform.

Long Term Cell Viability Studies—To study long-term viability of cells in the platform, HeLa cells cultured in DMEM/F12 media (Gibco, Gaithersburg, Md.) with 0.1% fetal bovine serum (Gibco, Gaithersburg, Md.) were loaded into the platform as previously described. Cells were maintained in the platform for predetermined durations of 12, hours 1, 2 4 and 7 days and periodically monitored using a microscope. Reproducibility was examined by repeating each experiment 6 times. At the end of the designated time period the valve on the input line to the platform was closed. Phosphate buffered saline (PBS) containing 5 μl/ml Vybrant DiO cell-labeling solution (V-22886) (Molecular Probes, Eugene, Oreg.) and 1 μg/ml propidium iodide (Molecular Probes, Eugene, Oreg.) was injected to the platform via the syringe injection port and cells incubated for a further 20 minutes at 37° C. prior to viewing under a fluorescent microscope. Vybrant DiO cell-labeling solution (V-22886) is a cell tracking dye with cells incorporating the label fluorescing yellow green. Propidium iodide is a molecule that enters cells either when cells are electroporated or when they are dead with cells incorporating propidium iodide fluorescing red. Fluorescence intensity of the HeLa cells was assessed using a Zeiss Axiovert microscope (Carl Zeiss, Inc. Thornwood, N.Y.) equipped with epifluorescence, FITC and Texas red excitation filters and DAPI/FITC/T×Red emission filters (Chroma, Technology Corp., Rockingham, Vt.). Images were collected using Universal Imaging's Metamorph software (Universal Imaging Corp., Downington, Pa.) and a Photometrics CoolSnap HQ camera (Photometrics, Tuscon, Ariz.).

Reuse of Arrays (Cleaning Studies)—Previously used arrays were rinsed in concentrated sulfuric acid (Sigma, St. Louis Mo.) and hydrogen peroxide (Sigma, St. Louis, Mo.), or washed in an ultrasonic bath of 2% Hellmanex (Fisher Scientific, Fairlawn, N.J.) in water for periods of 15, 30 and 60 minutes. After washing, arrays were sonicated in ultra pure water (for 2 min.) to remove the cleaning solution then dried under a stream of nitrogen. Viability of cells in the arrays cleaned with Hellmanex was examined using the same procedure described in the long-term cell viability studies.

Delivery of Biological Molecules to Individual Cells: Cy5-hybrid uptake—Raji cells were incubated in RPMI-1640 media (Gibco, Gaithersburg, Md.) supplemented with 10% heat-inactivated fetal bovine serum (FBS) (Sigma, St. Louis, Mo.) in a 37° C. incubator containing 5% CO₂. Cy5-modified RNA:DNA siHybrid molecules targeted against the green fluorescent protein (GFP) mRNA sequence were designed using Dharmacon's siDesign software (www.dharmacon.com). The RNA antisense sequence was 5′-UUGUCGGCCAUGAUAUAGAdTdT-3′ and was purchased from Dharmacon (Lafayette, Colo.). The DNA sense strand, 5′-TCTATATCATGGCCGACAATT-3′was labeled at the 5′ end with Cy5 and was purchased from Sigma-Genosys (The Woodlands, Tex.). Lyophilized pellets were resuspended at 1 μg/μl in nuclease-free, 10 mM Tris pH 8.0 (Ambion, Austin, Tex.). Equimolar concentrations of sense and antisense strands were mixed and annealed by incubating at 95° C. for 5 minutes followed by a slow cool of −0.1° C./s to 37° C. and a 1 hour hold at 37° C. The samples were then slow cooled to 4° C. and held until removed. Cells were resuspended at a concentration of 10⁶ cell/ml and Cy5-sihybrid added to a final concentration of 120 nM. The suspension was injected into the platform via the syringe injection port. Imaging was performed using a Zeiss Axiovert 200 microscope equipped with reflected light differential interference contrast (DIC), epi-fluorescence, a Cy5 filter set and a Zeiss Axiocam HRM high resolution digital camera. Cell capture in individual wells was monitored using DIC. Fluorescence intensity of Cy5 within cells was determined using Universal Imaging's Metamorph software (Universal Imaging Corp, Downington, Pa.).

Efficiency of Cell Capture—The arraying capability of the platform was illustrated showing a fluorescent image of arrayed HeLa cells that were stained with the fluorescent 5 μl/ml Vybrant DiO cell-labeling solution (V-22886) and 1 μg/ml propidium iodide. The yellow green color indicated that the cells were viable. Cell arraying rates showed a great degree of variability from as low as 5% to as high as 87% of the wells capturing cells during loading. The initial concentration of cells injected into the platform appears to have a large bearing on the loading efficiency, presumably because it has a direct effect on the concentration of cells in the flow channels. Applicants found that injected cell concentrations of ˜10⁶ cells/ml produce optimal loading using this chip design. Injected cell concentrations of ˜10⁵ cells/ml tend to produce poor loading efficiencies of less than 10% while injected cell concentrations of 10⁷ cells/ml tend to produce blockages or restrictions that impeded media flow in the array. Applicants observed that cells sometimes seen stuck to the main channel surface probably due to cell surface protein interactions with the channel surfaces and that this effect increases with increasing injected cell concentration. For injected cell concentrations of 10⁶ cells/ml we typically obtained well loading efficiencies of between 36 and 56%.

In addition, several other factors that are not thoroughly understood affect the fill process including specific array geometry that governs the movement of the cells through the array. Effective cell concentration near the capture wells could be increased with a reduction in the width of the main flow channel. Experiments conducted using an array with flow channels of width 250 μm (i.e., half the width of Applicants standard flow channels) indicated a 50% increase in cell capture rates.

The depth of the microchannels in the back wall of each well also appears to affect loading efficiency. Deeper microchannels leading to reduced pressure drop across wells were conducive to better fill rates. However, increased depth of the microchannels resulted in increased cell deformation and lysing, as the cells attempt to follow the flow into the microchannels. Smaller microchannels with a corresponding higher-pressure drop limited this cell deformation, as do longer secondary channel lengths. However, such geometries that limit cell distortion also resulted in a decreased fill percentage. As the goal was to affect the cells as little as possible by the mechanical processes, a lower loading efficiency was acceptable. Cell capture wells with several narrow microchannels may help increase loading efficiency while minimizing cell deformation and lysing.

Long-term Viability—Viability experiments indicated that the platform could effectively maintain HeLa cells for up to 7 days with greater than 95% of cells remaining in the array wells viable. Periodic cell monitoring indicated that some of the cells seemed to vanish after a period of a few days. Cell lysing was unlikely to be the cause of this, as no cellular debris was visible in the vacated capture wells. We believe that gas bubbles in the media are a plausible cause of this phenomenon. Bubbles were frequently observed in the media within the array. Cells were sometimes observed at the media-gas interface of these bubbles and it is presumed that cells may have attached to the bubble meniscus and been swept away by the media flow. To remedy this, gas-permeable filters will be added to future versions of the chip. However, this was an infrequent problem, and did not significantly alter the loaded cell fraction.

Cleaning Studies—While microfabricated chips are inexpensive when produced in bulk, it is convenient to be able to reuse them. Typically, cellular debris and other biologic material was visible in the flow channels, cell capture wells and suction ports of a chip after it had been used to array cells. Cleaning these used arrays with acid and hydrogen peroxide was ineffective, with much cellular debris remaining in the flow channels. However, cleaning the arrays by ultrasonic washing in 2% Hellmanex for 15 minutes removed cell debris, protein deposits and buffers from capture wells and suction ports. Cleaning for extensive periods of time (30 min to 1 hr) proved deleterious to the arrays and channel etching was noted. Arrays cleaned in 2% Hellmanex for 15 minutes were reusable with HeLa cells remaining viable in the reused arrays for at least 24 hours (limit of Applicants testing).

Delivery of Biological Molecules to Individual Cells—The array is ideally suited to single cell pharmacokinetic studies. In this vein Applicants group is using the platform to study delivery of gene silencing agents in individual cells. One key question in gene silencing is the question of the degree of gene shutdown. If population-based assays show a 75% knockdown, for example, there is the question of whether 75% of the cells were completely silenced, and the remaining cells were unaltered, or whether all of the cells had the function of the particular gene reduced by 75%. Knowing if the molecules entered each cell would give insight into this problem. A specific problem in Applicants group is the uptake of small RNA:DNA hybrids into mammalian cells. RNA:DNA siHybrid-based gene silencing has been shown to reduce gene expression in mammalian cell cultures. Applicants used the array to study uptake of siHybrids in individual cells in order to gain insights into and improve delivery of the constructs to target cells. Relative fluorescence intensity remained near background levels for the first 2 h. Cells then began to show increasing Cy5 intensity up to a maximum at 6-8 h. Applicants found that curves of fluorescence intensity versus time can vary widely between individual cells suggesting that uptake of the sihybrids may not be uniform across the cell population. The variation in fluorescent intensity was also observed between cells when pools of cells are dosed with siHybrids and maintained in conventional well plates.

Mechanical Arraying Technique—A major advantage of using mechanical techniques to locate cells in specific locations within the array is the universality of the technique to a variety of cell types and media compositions. Conversely, adhesion patches work best for cells with specific surface properties conducive to attachment while dielectrophoresis relies on specific electrical properties of the cell and the media. A second advantage to Applicant's platform is its ability to capture a single isolated cell in a multiple of specific locations. Although other techniques, such as optical tweezers, are capable of trapping a single cell, multiple capture sites can be difficult to achieve, while dielectrophoretic devices can be hindered by trapping multiple cells at each capture zone. Additionally, other approaches may have more difficulty in trapping a single cell out of a flowing system.

A key issue with the mechanical arraying techniques is avoidance of excessive force on the cell while achieving an easily manipulated fluid flow field. Cells are highly deformable and are able to squeeze into relatively small openings. Higher viscosity solutions require larger channel cross-sections for reasonable flow rates and reasonable pressure drops. Directing transient flows in arrays through manipulation of external flow control devices such as valves and pumps can prove difficult due to fluid capacitances established due to flexible tubing and bubbles.

An advantage of these chips is that while the complete system is fairly standardized, different arraying surfaces can easily be made and used with the same system. Applicants have made different versions of this for collaborators, each designed to array and maintain cells in configurations of the end user's needs. For example Applicants are developing a cell capture array made entirely of glass to eliminate the need for reflected light DIC. Applicants are also currently developing a similar system to array bacterial cells, which will also be modifiable to a variety of configurations. These viable-cell arrays will allow high-throughput experiments in cellular and pharmacokinetics to be combined with the statistical precision of single-cell data acquisition.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

The invention claimed is:

1. An apparatus for capturing a cell for analysis, comprising:
a capture structure,
a flow channel in said capture structure,
a capture well in said capture structure that is operatively connected to said flow channel,
a fluid operatively located in said flow channel and said capture well,
a small hole in said capture well that is large enough for said fluid to flow through said capture well but smaller than the cell so that the cell is captured and located in said capture well for analysis, and
a system for applying suction to said, wherein said fluid carries the cell so that the cell is captured in said capture well for analysis.

2. The apparatus for locating a cell for analysis of claim 1 wherein said capture well is located in said capture structure in a horizontal arrangement.

3. The apparatus for locating a cell for analysis in a vertical format of claim 1 wherein said capture well is located in said chip in a vertical arrangement.

4. The apparatus for locating a cell for analysis of claim 1 including a probe in said flow channel proximate said capture well.

5. The apparatus for locating a cell for analysis of claim 4 wherein said probe is coated with metal.

6. The apparatus for locating a cell for analysis of claim 4 wherein said probe is functionalized.

7. The apparatus for locating a cell for analysis of claim 1 including a needle in said flow channel proximate said capture well.

8. An apparatus for locating a cell for analysis, comprising:
a chip,
a capture well extending through said chip,
a system for applying suction to said capture well,
a fluid,
a fluid flow channel in said chip leading to said capture well, and
a small hole in said capture well that is large enough for said fluid to flow through said capture well but smaller than the cell so that the cell is captured and located in said capture well for analysis.

9. The apparatus for locating a cell for analysis of claim 8 wherein said capture well is located in said capture structure in a horizontal arrangement.

10. The apparatus for locating a cell for analysis in a vertical format of claim 8 wherein said capture well is located in said chip in a vertical arrangement.

11. An apparatus for locating a cell for analysis, comprising:
a chip,
capture well means extending through said chip,
means for applying suction to said capture well,
a fluid, and
a fluid flow channel leading to said capture well,
wherein said capture well means includes a small hole large enough for said fluid to flow through said capture well but smaller than the cell so that the cell is captured and located in said capture well for analysis.

12. The apparatus for locating a cell for analysis of claim 11 wherein said capture well is located in said capture structure in a horizontal arrangement.

13. The apparatus for locating a cell for analysis in a vertical format of claim 11 wherein said capture well is located in said chip in a vertical arrangement.

14. A method of locating a cell for analysis, comprising the steps of:
providing a chip, a capture well extending through said chip that includes a small hole large enough for said fluid to flow through said capture well but smaller than the cell, a fluid, and a fluid flow channel leading to said capture well; and
providing fluid flow along said fluid flow channel and through said capture well in said chip, wherein the cell flows with the fluid along said flow channel into the capture well but no further.

15. The method of locating a cell for analysis in a vertical format of claim 14, wherein said step of providing fluid flow along said fluid flow channel and through said capture well in said chip is created by suction.

16. The method of locating a cell for analysis in a vertical format of claim 14, wherein said step of providing fluid flow along said fluid flow channel and through said capture well in said chip results in the cell being aspirated onto the small hole which is used to locate it in the cell.

17. A method of locating a cell for analysis, comprising the steps of:
providing a capture structure, a flow channel in said capture structure,
a capture well in said flow channel, and a fluid; and
providing fluid flow along said fluid flow channel and through said capture well, wherein the cell flows with the fluid along said flow channel into the capture well but no further.

18. The method of locating a cell for analysis of claim 17, wherein said step of providing fluid flow along said fluid flow channel and through said capture well is created by suction.

19. The method of locating a cell for analysis in a vertical format of claim 17, wherein said step of providing fluid flow along said fluid flow channel and through said capture well results in the cell being aspirated onto capture well which is used to locate the cell.

20. The method of locating a cell for analysis in a vertical format of claim 17, wherein said step of providing fluid flow along said fluid flow channel and through said capture well is completed in a horizontal arrangement.

21. The method of locating a cell for analysis in a vertical format of claim 17, wherein said step of providing fluid flow along said fluid flow channel and through said capture well is completed in a vertical arrangement.
